(19) United States
(12) Patent Application Publication
(10) Pub. No.: US 2024/0387032 A1
(43) Pub. Date: Nov. 21, 2024

(54) AUTOMATIC ADJUSTMENT OF MEASUREMENT INTERVAL TIMES FOR PHYSIOLOGICAL PARAMETERS

(71) Applicant: Dragerwerk AG & Co. KGaA, Lubeck (DE)

(72) Inventors: Ryan Forde, Salem, NH (US); Devin Weidner, Andover, MA (US)

(21) Appl. No.: 18/658,536

(22) Filed: May 8, 2024

Related U.S. Application Data
(60) Provisional application No. 63/467,433, filed on May 18, 2023.

(51) Int. Cl. G16H 40/40; G16H 40/63

(52) U.S. Cl. CPC G16H 40/40; G16H 40/63

(57) ABSTRACT

A medical system that is configured to automatically generate customized measurement schedules is described. The system may include one or more sensors connected to the medical system and configured to measure patient parameters of a patient. The system may further include a memory to store one or more programs that are executed by medical device and a display for displaying the measured patient parameters. The programs may execute a predefined measurement schedule to measure the patient parameters of the patient automatically generate a modified measurement schedule automatically in response to a modification event. Said modification event causing the system to then adjust the measurement schedule in response to the modification event.

### CROSS REFERENCE TO RELATED APPLICATIONS

The present application claims priority to and the benefit of U.S. Prov. Pat. App. Ser. No. 63/467,433, which was filed on May 18, 2023, for all purposes, including the right of priority, which application is hereby incorporated herein by reference in its entirety and to the extent that is not inconsistent with the present disclosure.

### TECHNICAL FIELD

In general, the present disclosure relates to the field of physiological parameter monitoring. More specifically, the present disclosure relates to the automatic adjustment of measuring intervals based on previously measured physiological parameters, such as non-invasive blood pressure.

### BACKGROUND

Blood pressure measurement, such as non-invasive blood pressure, is a test that measures the force or pressure in a person's arteries as their heart pumps. Blood pressure is measured and presented as two numbers referred to as Systolic pressure, which is the pressure inside your arteries when the heart beats, and Diastolic pressure, which is the pressure inside the artery when the heart rests between beats. Blood pressure monitoring is an important part of patient monitoring because helps to provide a picture of the patient's health and their risk of cardiac issues such as heart disease or stroke.

In various medical facilities, clinicians often measure a patient's NIBP at regular intervals. These measurements can be performed manually by clinicians or automatically with an automated blood pressure monitoring system. Likewise, the frequency of those measurements (i.e., the intervals) is often determined by clinicians and is guided by the patient's condition and/or protocols of the medical facility. For example, depending on the medical condition of the patient, the interval could be as short as every 3 minutes or possibly as long as every 1 hour. Similarly, in some care environments, the measurement interval could be as long as every 4 hours, 8 hours, or even 24 hours or more, in some scenarios.

### SUMMARY

Currently, some features exist and are integrated into patient monitors to help medical personnel during patient care. One such feature is an alarm limit, which is typically triggered in response to a physiological parameter of the patient exceeding a predefined threshold. Some examples of thresholds include heart rates that are too fast or too slow, oxygen saturation levels being below a predefined percentage, or blood pressure levels being too low or too high, to list a few examples. One issue with alarm limits is that thresholds for triggering the alarms are typically either in a state of activation or not. That is, a threshold is either, “exceeded” or “not exceeded.” For example, if an alarm threshold is configured to trigger when a patient's heart rate falls below 60 beats per minute, it does not matter whether the heart rate is 75 or 61. The alarm will not trigger until the heart rate falls below 60. To counter this problem, many systems implement trend monitoring, which is the process of monitoring parameters over time to determine if a change is occurring over time. A problem with trend monitoring is that the scheduling of the measurements is not adaptive to changing conditions of the patient.

Accordingly, it would be advantageous to provide an efficient and useful way to automatically generate physiological measurement schedules for patients that fulfill different clinical needs based on patient characteristics such as physiological information, medical condition, patient parameters, and further based on a patient's location within a medical facility, for example. Likewise, the schedule for measuring parameters could automatically adjust the interval times of the measurements in response to changing patient conditions. This would help to identify potential medical problems sooner, reduce stress and cognitive load on clinicians, support more rapid patient assessment and accurate clinical documentation, and improve overall patient care.

The present disclosure provides an electronic device capable of executing an automatically adjustable physiological measurement schedule for measuring one or more physiological parameters of a patient. As used herein, the term “automatically” and its derivatives means under electronic, programmed control and without human intervention or participation. The electronic device includes a display configured to display medical information related to a patient including physiological data, a memory configured to store one or more programs, and one or more processors configured to execute the one or more programs.

The one or more programs when executed by the one or more processors provides a graphical user interface (GUI) on the display. The GUI includes a customizable measurement schedule for the patient with one or more selections. Moreover, the one or more programs also automatically generate modified measurement schedules of the one or more physiological parameters for the patient in response to changing physiological parameters.

In an embodiment of the present disclosure, the customizable measurement schedule is directed to discrete or continuous measurements of non-invasive blood pressure (NIBP). However, it is understood that such the features of the present disclosure could also be applied to other patient parameters such as temperature, heart rate, electrocardiogram (ECG), non-invasive peripheral oxygen saturation (SpO2), end tidal carbon dioxide (etCO2), apnea of the patient, neuromuscular transmission (NMT), and cardiac output (CO), and electroencephalogram (EEG), to list a few examples.

In some embodiments, a medical device configured to automatically generate customized measurement schedules comprises one or more sensors attached to a patient configured to measure patient parameters of the patient, a memory configured to store one or more programs that are executed by one or processors of the medical device, and a display configured to display the measured patient parameters. The one or more processors maybe be communicatively connected to the memory and configured to execute the one or more programs in order to execute a predefined measurement schedule. The medical device may be further configured to configured to activate the one or more sensors in order to measure the patient parameters of the patient, receive the measured patient parameters, transmit the measured patient parameters to memory and to the display, and generate a modified measurement schedule automatically in response to a modification event. The medical device may further implement the modified measurement schedule, and provide an indication that the modified measurement schedule has been generated and implemented.

The medical device may further implement at least one of a change in a medical condition of the patient, a change in a location of the patient, a change in personal information of the patient, and a change in physiological information of the patient. The change in the medical condition of the patient may include at least one of detection hypovolemia, sepsis, cardiac events, and shock. The change in the location of the patient may include at least one of the patient moving to an emergency room, an operating room, an intensive care unit, a neonatal intensive care unit, a post anesthesia care unit, a recovery room, and a labor and delivery room. Additionally, the change in personal information of the patient may include obtaining information about at least one of current medications taken by or administered to the patient, information about the patient's medical history, and personal information including information about the patient's age, height, weight, and/or gender.

The medical device may further implement a change in response to medication taken by the patient and the medications taken by, or administered to, the patient may include anesthesia, vasoactive medications, antiemetics, and pain medications.

The modification event described herein may be based on a percentage change in one or more of the measured patient parameters. Additionally, the modification event may be based on one or more of the measured patient parameters being within a predefined percentage of an alarm limit. In some embodiments, the patient parameters of the patient may include at least one of non-invasive blood pressure (NIBP), temperature, heart rate, an electrocardiogram (ECG), non-invasive peripheral oxygen saturation (SpO2), end tidal carbon dioxide (etCO2), apnea of the patient, neuromuscular transmission (NMT), and cardiac output (CO).

In some embodiments, the medical device may provide a notification that a modified measurement schedule has been automatically generated and implemented, which includes providing a notification by at least one of an electronic mail, a text message, a page message sent to a pager, a notification being transmitted to a central monitoring station, and an alert displayed on the medical device. The notification may include at least one of a visual and an audible notification indicating generation and implementation of the modified measurement schedule.

The medical device may implement one or more processors, which store in the memory, a time, a date, and the modification event that caused the generation and implementation of the automatically generated modified measurement schedule. Likewise, the medical device may store in the memory each automatically generated modified measurement schedule.

The medical device may further permit a user to override the automatically generated modified measurement schedule, permit a user to select any previous automatically generated modified measurement schedule, and/or permit a user to override the automatically generated modified measurement schedule and return to the original predefined measurement schedule, or any previous automatically generated modified measurement schedule.

### BRIEF DESCRIPTION OF THE DRAWINGS

In the drawings, like reference numbers generally indicate identical, functionally similar, and/or structurally similar elements.

FIG. 1 is a schematic diagram of an example of a system capable of executing a customizable physiological measurement schedule for measuring physiological parameters according to an embodiment of the present disclosure;

FIG. 2 is a schematic diagram of an example of a physiological monitoring device capable of executing a customizable physiological measurement schedule for measuring physiological parameters according to an embodiment of the present disclosure;

FIG. 3 is a schematic diagram of an example of a system including a server/central computer according to an embodiment of the present disclosure;

FIG. 4 is a schematic diagram of an example of a server/central computer according to an embodiment of the present disclosure;

FIG. 5A, FIG. 5B, and FIG. 5C illustrate examples of graphical user interfaces (GUIs) for executing automatically generated modified measurement schedules for measuring physiological parameters according to embodiments of the present disclosure;

FIG. 6 illustrates and example of a graphical user interface (GUIs) for executing automatically generated modified measurement schedules for measuring physiological parameters according to embodiments of the present disclosure;

FIG. 7 illustrates and example of a graphical user interface (GUIs) for executing automatically generated modified measurement schedules for measuring physiological parameters according to embodiments of the present disclosure; and

FIG. 8 illustrates an example of a method and an algorithm for automatically generating modified measurement schedules for measuring physiological parameters according to an embodiment of the present disclosure.

### DETAILED DESCRIPTION

The following detailed description is made with reference to the accompanying drawings and is provided to assist in a comprehensive understanding of various example embodiments of the present disclosure. The following description includes various details to assist in that understanding, but these are to be regarded merely as examples and not for the purpose of limiting the present disclosure as defined by the appended claims and their equivalents. The words and phrases used in the following description are merely used to permit a clear and consistent understanding of the present disclosure. In addition, descriptions of well-known structures, functions, and configurations may have been omitted for clarity and conciseness. Those of ordinary skill in the art will recognize that various changes and modifications of the examples described herein can be made without departing from the spirit and scope of the present disclosure.

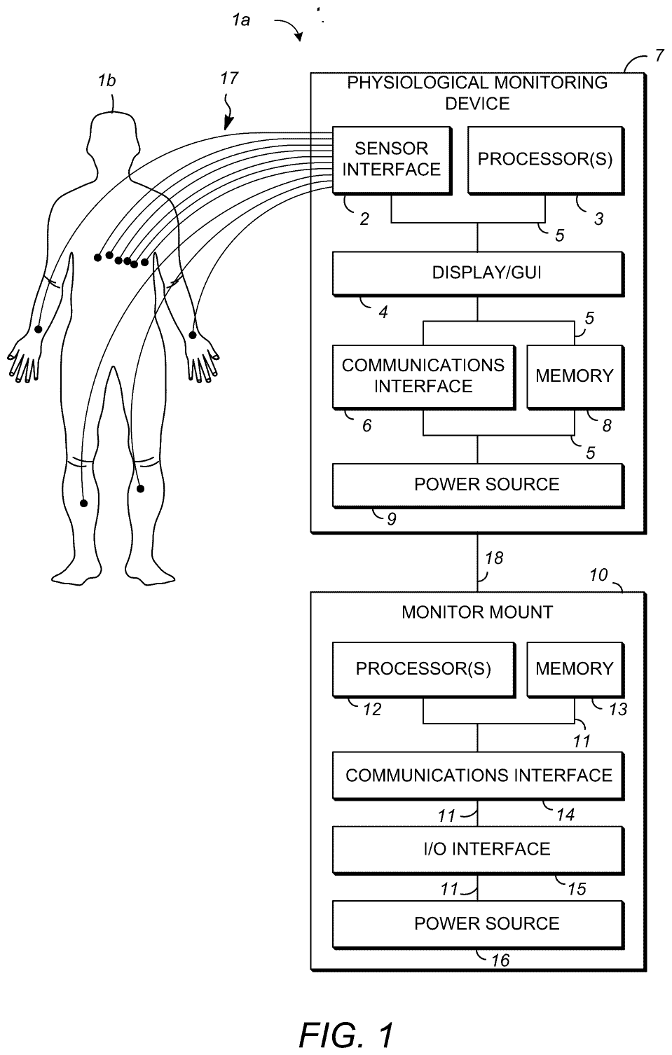

FIG. 1 is a schematic diagram of an example of a system 1a capable of executing a customizable physiological measurement schedule for measuring physiological parameters according to an embodiment of the present disclosure. As shown in FIG. 1, the system 1a includes an electronic device such as physiological monitoring device 7 (or, simply, a patient monitor) capable of receiving physiological data from various sensors 17 connected to a patient 1b, and a monitor mount 10, to which the physiological monitoring device 7 is removably mounted or docked.

In general, it is contemplated by the present disclosure that the physiological monitoring device 7 and the monitor mount 10 include electronic components and/or electronic computing devices operable to receive, transmit, process, store, and/or manage patient data and information associated with performing the functions of the system 1a, which encompasses any suitable processing device adapted to perform computing tasks consistent with the execution of computer-readable instructions stored in a memory or a computer-readable recording medium.

Further, any, all, or some of the computing devices in the physiological monitoring device 7 and the monitor mount 10 may be adapted to execute any operating system, including Linux, UNIX, Windows Server, etc., as well as virtual machines adapted to virtualize execution of a particular operating system, including customized and proprietary operating systems. The physiological monitoring device 7 and the monitor mount 10 are further equipped with components to facilitate communication with other computing devices over one or more network connections, which may include connections to local and wide area networks, wireless and wired networks, public and private networks, and any other communication network enabling communication in the system 1a.

As shown in FIG. 1, the physiological monitoring device 7 is, for example, a portable or stationary patient monitor implemented to monitor various physiological parameters of the patient 1b via the sensors 17. The physiological monitoring device 7 includes a sensor interface 2, one or more processors 3, a display 4 including a graphical user interface (GUI), a communications interface 6, a memory 8, and a power source 9. The sensor interface 2 can be implemented in software or hardware and used to connect via wired and/or wireless connections to one or more physiological sensors 17 for gathering physiological data from the patient 1b.

The data signals from the sensors 17 include, for example, data related to an electrocardiogram (ECG), non-invasive peripheral oxygen saturation (SpO2), non-invasive blood pressure (NIBP), temperature, and/or end tidal carbon dioxide (etCO2), apnea detection, neuromuscular transmission (NMT), and cardiac output (CO), or other similar physiological data that can be measured discretely or continuously. The one or more processors 3 are used for controlling the general operations of the physiological monitoring device 7.

The display 4 is for displaying various patient data, measurement schedules, and hospital or patient care information and for allowing communication between a user and the physiological monitoring device 7. The display 4 may include, but is not limited to, a pointing device, a keyboard, a liquid crystal display (LCD), thin film transistor (TFT), light-emitting diode (LED), high definition (HD), or other similar GUI with touch screen capabilities, none of which are separately shown. The patient information displayed can, for example, relate to the measured physiological parameters of the patient 1b (e.g., blood pressure, heart-related information, pulse oximetry, respiration information, etc.) as well as information related to a customizable measurement schedule for taking the physiological parameters of the patient 1b.

The communications interface 6 allows the physiological monitoring device 7 to directly or indirectly (via, for example, the monitor mount 10) communicate with one or more computing networks and devices. The communications interface 6 can include various network cards, interfaces, or circuitry to permit wired and wireless communications with such computing networks and devices. The communications interface 6 can also be used to implement, for example, a Bluetooth connection, a cellular network connection, and/or a WIFI® connection. Other wireless communication connections implemented using the communications interface 6 include wireless connections that operate in accordance with, but are not limited to, IEEE802.11 protocol, a Radio Frequency For Consumer Electronics (RF4CE) protocol, ZigBee protocol, and/or IEEE802.15.4 protocol.

Additionally, the communications interface 6 can permit direct (i.e., device-to-device) communications (e.g., messaging, signal exchange, etc.) such as from the monitor mount 10 to the physiological monitoring device 7 using, for example, a USB connection. The communications interface 6 can also permit direct device-to-device connection to other devices such as to a tablet, PC, or similar electronic device, or to an external storage device or memory.

The memory 8 can be used to store any type of instructions, patient data, and measurement schedules associated with algorithms, processes, or operations for controlling the general functions and operations of the physiological monitoring device 7.

The power source 9 can include a self-contained power source such as a battery pack and/or include an interface to be powered through an electrical outlet (either directly or by way of the monitor mount 10). The power source 9 can also be a rechargeable battery that can be detached allowing for replacement. In the case of a rechargeable battery, a small built-in backup battery (or super capacitor) can be provided for continuous power to be provided to the physiological monitoring device 7 during battery replacement. Communication between the components of the physiological monitoring device 7 (e.g., 2, 3, 4, 6, 8, and 9) are established using an internal bus 5.

As shown in FIG. 1, the physiological monitoring device 7 is connected to the monitor mount 10 via a connection 18 that establishes a communication connection between, for example, the respective communications interfaces 6, 14 of the devices 7, 10. The connection 18 permits the monitor mount 10 to detachably secure the physiological monitoring device 7 to the monitor mount 10. In this regard, “detachably secure” means that the monitor mount 10 can secure the physiological monitoring device 7, but the physiological monitoring device 7 can be removed or undocked from the monitor mount 10 by a user when desired. The connection 18 may include, but is not limited to, a universal serial bus (USB) connection, parallel connection, a serial connection, coaxial connection, a High-Definition Multimedia Interface (HDMI) connection, or other similar connection known in the art connecting to electronic devices. Additionally, the connection may include optical communications interfaces and/or high-speed wireless communication interfaces.

The monitor mount 10 includes one or more processors 12, a memory 13, a communications interface 14, an I/O interface 15, and a power source 16. The one or more processors 12 are used for controlling the general operations of the monitor mount 10. The memory 13 can be used to store any type of instructions associated with algorithms, processes, or operations for controlling the general functions and operations of the monitor mount 10.

The communications interface 14 allows the monitor mount 10 to communicate with one or more computing networks and devices (e.g., the physiological monitoring device 7). The communications interface 14 can include various network cards, interfaces, or circuitry to permit wired and wireless communications with such computing networks and devices. The communications interface 14 can also be used to implement, for example, a Bluetooth connection, a cellular network connection, and a WIFI® connection. Other wireless communication connections implemented using the communications interface 14 include wireless connections that operate in accordance with, but are not limited to, IEEE802.11 protocol, a Radio Frequency For Consumer Electronics (RF4CE) protocol, ZigBee protocol, and/or IEEE802.15.4 protocol.

The communications interface 14 can also permit direct (i.e., device-to-device) communications (e.g., messaging, signal exchange, etc.) such as from the monitor mount 10 to the physiological monitoring device 7 using, for example, a USB connection, coaxial connection, or other similar electrical connection. The communications interface 14 can permit direct (i.e., device-to-device) to other devices such as to a tablet, PC, or similar electronic device, or to an external storage device or memory.

The input/output (I/O) interface 15 can be an interface for enabling the transfer of information between the monitor mount 10, one or more physiological monitoring devices 7, and external devices such as peripherals connected to the monitor mount 10 that need special communication links for interfacing with the one or more processors 12. The I/O interface 15 can be implemented to accommodate various connections to the monitor mount 10 that include, but are not limited to, a universal serial bus (USB) connection, parallel connection, a serial connection, a coaxial connection, a High-Definition Multimedia Interface (HDMI) connection, or other known connection in the art connecting to external devices.

The power source 16 can include a self-contained power source such as a battery pack and/or include an interface to be powered through an electrical outlet (either directly or by way of the physiological monitoring device 7). The power source 16 can also be a rechargeable battery that can be detached allowing for replacement. Communication between the components of the monitor mount 10 (e.g., 12, 13, 14, 15, and 16) are established using an internal bus 11.

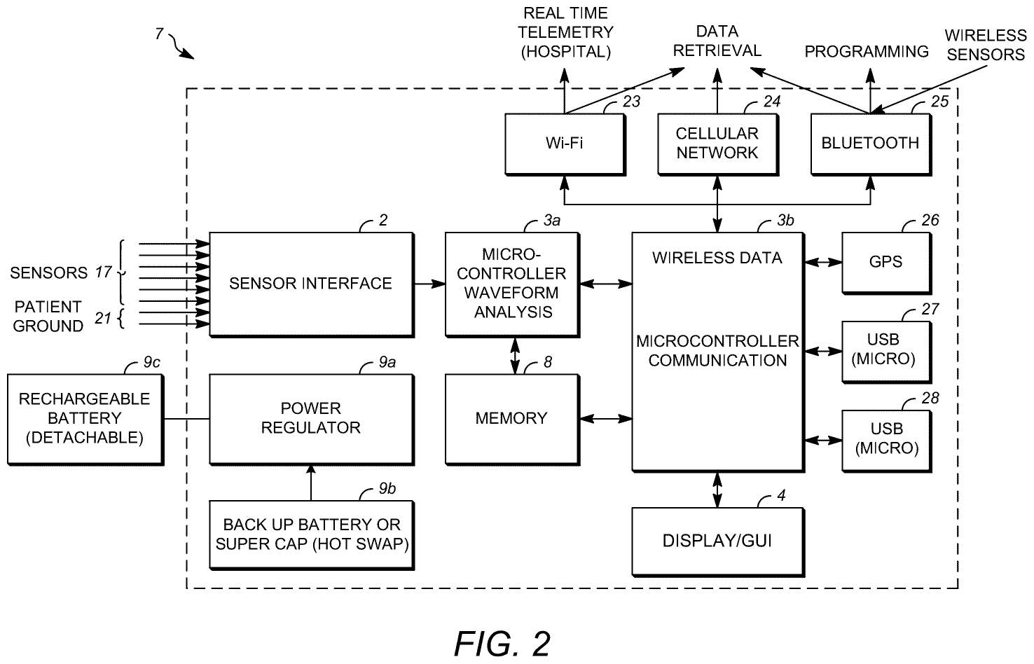

FIG. 2 is a schematic diagram of an example of a physiological monitoring device capable of executing a customizable physiological measurement schedule for measuring physiological parameters according to an embodiment of the present disclosure.

As shown in FIG. 2, the physiological monitoring device 7 is, in this particular embodiment, attached to several different types of sensors 17 (including electrodes or other similar devices) known in the art for gathering physiological data related to the patient 1b (e.g., as shown on the left side of FIG. 1). The sensors 17 are communicatively coupled to physiological monitoring device 7 by, for example, a wired connection input to the sensor interface 2. It is contemplated by the disclosure that the physiological monitoring device 7 can also be connected to other wireless sensors using the communication interface 6, which includes circuity for receiving data from and sending data to one or more devices using, for example, a Bluetooth connection 25. The communications interface 6 shown in FIG. 1 is represented in FIG. 2 by the combination of microcontroller 3b and elements 23-28.

The data signals from the sensors 17 received by the physiological monitoring device 7 include data related to, for example, an ECG, SpO2, NIBP, temperature, and/or etCO2. The data signals received from an ECG sensor and the SpO2 sensor can be analog signals. The data signals for the ECG and the SpO2 are input to the sensor interface 2, which can include an ECG data acquisition circuit and a SpO2 data acquisition circuit. Both the ECG data acquisition circuit and the SpO2 data acquisition circuit include amplifying and filtering circuity as well as analog-to-digital (A/D) circuity that convert the analog signal to a digital signal using amplification, filtering, and A/D conversion methods known in the art.

As another example, the data signals related to NIBP, temperature, and etCO2 can be received from sensors 17 to the sensor interface 2, which can include a physiological parameter interface such as serial interface circuitry for receiving and processing the data signals related to NIBP, temperature, and etCO2. The ECG data acquisition circuit, an SpO2 data acquisition circuit, and a physiological parameter interface are described as part of the sensor interface 2. However, it is contemplated by the present disclosure that the ECG data acquisition circuit, the SpO2 data acquisition circuit, and the physiological parameter interface can be implemented as circuits separate from the sensor interface 2.

The processing performed by the ECG data acquisition circuit, the SpO2 data acquisition circuit, and external physiological parameter interface produces digital data waveforms that are analyzed by the microcontroller 3a. The processors 3 shown in FIG. 1 are represented in FIG. 2 as microcontrollers 3a and 3b. The microcontroller 3a, for example, analyzes the digital waveforms to identify certain digital waveform characteristics and threshold levels indicative of conditions (abnormal and normal) of the patient 1b using methods known in the art. The microcontroller 3a includes a memory or uses the memory 8.

The memory stores software or algorithms with executable instructions and the microcontroller 3a can execute a set of instructions of the software or algorithms in association with executing different operations and functions of the physiological monitoring device 7 such as analyzing the digital data waveforms related to the data signals from the sensors 17. The results of the operations performed by the microcontroller 3a are passed to the microcontroller 3b. The microcontroller 3b includes a memory or uses the memory 8.

As noted above, in FIG. 2, the communication interface 6 shown in FIG. 1 is represented by the combination of microcontroller 3b and elements 23-28. For example, the microcontroller 3b includes communication interface circuitry for establishing communication connections with various devices and networks using both wired and wireless connections, and transmitting physiological data, patient and transport information (e.g., transport times and patient location information), results of the analysis by the microcontroller 3a, and alerts and/or alarms to the patient 1b, clinicians and/or caregivers. The memory 8 stores software or algorithms with executable instructions and the microcontroller 3b can execute a set of instructions of the software or algorithms in association with establishing the communication connections.

As shown in FIG. 2, wireless communication connections established by the communication interface circuity of microcontroller 3b include a Bluetooth connection 25, a cellular network connection 24, and a WIFI® connection 23. The wireless communication connections can allow, for example, patient and hospital information, alerts, and physiological data to be transmitted in real-time within a hospital wireless communications network (e.g., WIFI®) as well as allow for patient and hospital information, alerts, and physiological data to be transmitted in real-time to other devices (e.g., Bluetooth 25 and/or cellular networks 24).

It is also contemplated by the present disclosure that the communication connections established by the microcontroller 3b permit communications over other types of wireless networks using alternate hospital wireless communications such as wireless medical telemetry service (WMTS), which can operate at specified frequencies (e.g., 1.4 GHZ). Other wireless communication connections can include wireless connections that operate in accordance with, but are not limited to, IEEE802.11 protocol, a Radio Frequency For Consumer Electronics (RF4CE) protocol, ZigBee protocol, and/or IEEE802.15.4 protocol.

The Bluetooth connection 25 can also be used to provide the transfer of data to a nearby device (e.g., tablet) for review of data and/or changing of operational settings of the physiological monitoring device 7. The microcontroller 3b of the physiological monitoring device 7 provides a communication connection by direct wired (e.g., hard-wired) connections for transferring data using, for example, a USB connection 27 to a tablet, PC, or similar electronic device (not shown); or using, for example, a USB connection 28 to an external storage device or memory. Additionally, the microcontroller 3b includes a connection to a display 4 including a GUI for displaying patient information, physiological data or measured data, measurement schedules, alerts or alarms for the patient, clinicians and/or caregiver's information. Although the physiological monitoring device 7 is described in FIG. 1 as having two microcontrollers 3a and 3b, it is contemplated by the disclosure of the present application that one microcontroller can be implemented to perform the functions of the two microcontrollers 3a and 3b.

The display 4 may include, for example, a liquid crystal display (LCD), thin film transistor (TFT), light-emitting diode (LED), high definition (HD), or other similar GUI with touch screen capabilities. The display 4 also includes a GUI that provides a means for inputting instructions or information directly to the physiological monitoring device 7. As shown in FIG. 2, the physiological monitoring device 7 includes a global positioning system (GPS) or other location data system 26 that can be connected to the communication interface circuity of microcontroller 3b so that the physiological monitoring device can transmit to the clinician, caregiver, or other devices the location of the patient 1b at all times including the location of the patient 1b. Additionally, the location of the patient 1b can be used by the microcontroller 3b to determine an estimated time of arrival of the patient 1b.

For example, location data provided by the location data system 26, which may include information on a floor level, can be compared to stored information related to a hospital layout or a hospital map as well as information related to a patient's scheduled care (e.g., treatment or procedure scheduled for the patient 1b in a patient care area within the hospital). Based on the comparison results, the microcontroller 3b can determine the estimated time of arrival of the patient 1b to the patient care area within the hospital. The estimated time of arrival can be transmitted by the communication interface circuity of microcontroller 3b to, for example, the hospital wireless communications system.

Additionally, if it is determined by the microcontroller 3b that the patient 1b is not within the vicinity of the hospital wireless communications system (e.g., based on input from the location data system 26), the pertinent physiological data can be recorded and stored in the memory 8. Additionally, if the Bluetooth connection 25 or WIFI® connection 23 are not available (e.g., out of transmission range or not operable), then the microcontroller can store the physiological data in the memory 8 for later transmission when the Bluetooth connection or WIFI® connection becomes available.

The power source 9 shown in FIG. 1 is represented by elements 9a-9c in FIG. 2. As shown in FIG. 2, the power can be supplied using a rechargeable battery 9c that can be detached allowing for replacement. The rechargeable battery 9c may be, for example, a rechargeable lithium-ion battery. Additionally, a small built-in backup battery 9b (or supercapacitor) is provided for continuous power to the physiological monitoring device 7 during battery replacement. A power regulator or regulation circuit 9a is provided between the rechargeable battery 9c and small backup battery 9b to control which battery provides power to the physiological monitoring device 7. The physiological monitoring device 7 also includes a patient ground connection 21. The patient ground connection 21 can be used as a ground for single ended unipolar input amplifiers (e.g., precordial leads), or as a ground for bipolar input amplifiers (e.g., limb leads). It is also contemplated by the present disclosure that the power regulator 9a can include a self-contained power source such as a battery pack and/or include an interface to be powered through an electrical outlet (either directly or by way of the monitor mount 10). Communication between the components of the physiological monitoring device 7 can be established using an internal bus similar to the internal bus 5 discussed with reference to FIG. 1.

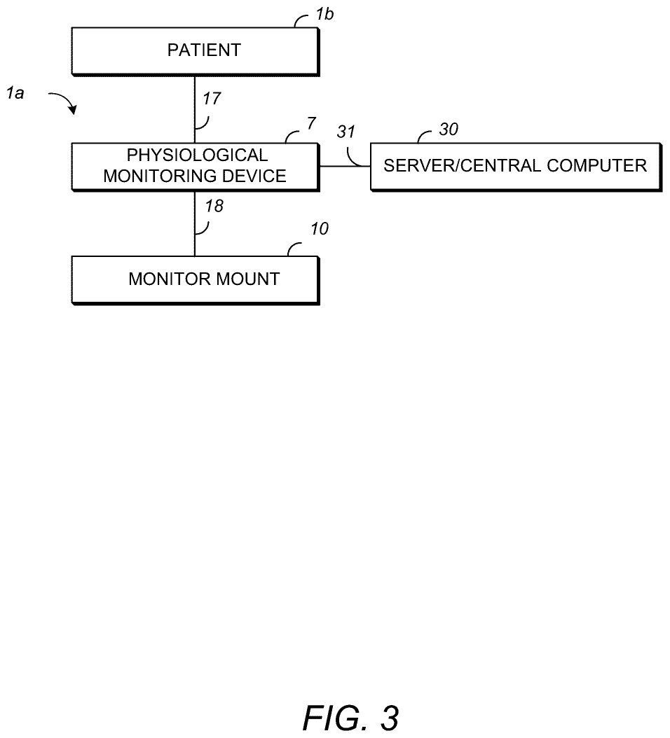

FIG. 3 is a schematic diagram of an example of a system 1a including a server/central computer according to an embodiment of the present disclosure. FIG. 3 includes the patient 1b, the physiological monitoring device 7, and the monitor mount 10 already discussed with reference to FIGS. 1 and 2. However, FIG. 3 also includes the addition of a server or central computer 30. As shown in FIG. 3, the physiological monitoring device 7 receives physiological data from various sensors 17 connected to the patient 1b, and the physiological monitoring device 7 is removably mounted or docked to the monitor mount 10. The physiological monitoring device 7 is connected to the monitor mount 10 via the connection 18 that establishes a communication connection between, for example, the respective communications interfaces 6, 14 of the devices 7, 10. The connection 18 permits the monitor mount 10 to detachably secure the physiological monitoring device 7 to the monitor mount 10.

The connection 18 may include, but is not limited to, a universal serial bus (USB) connection, parallel connection, a serial connection, coaxial connection, a High-Definition Multimedia Interface (HDMI) connection, or other similar connection known in the art for connecting to electronic devices. The physiological monitoring device 7 can also be connected to a server/central computer 30 via a wired or wireless connection 31 using the communication interface circuity of the communications interface 6 of the physiological monitoring device 7 described with reference to FIGS. 1 and 2. The server/central computer 30 can be located in or outside the hospital environment. For example, the server/central computer 30 can be located at a nurse station or other similar location within the hospital.

In one embodiment, the physiological monitoring device 7 may transmit, via the connection 31, physiological data collected by the sensors and/or other patient information (e.g., measurement schedules, patient location information, alert/alarm information) to the server/central computer 30 for storage and data processing. For example, upon the NIBP measurements with variable intervals configured by users on the physiological monitoring device 7, the NIBP data processed by the physiological monitoring device 7 along with related information may be transmitted and stored in the server/central computer 30.

In another embodiment, the server/central computer 30 may transmit control signals, via the connection 31, to control the functions of the physiological monitoring device 7 and the sensors that are connected to the device. As such, users are allowed to control the physiological measurements performed by the sensors or configure the measurement settings, via the user interface of the server/central computer 30. For example, the server/central computer 30 may allow users to configure NIBP measurements (e.g., customize measurement intervals and/or frequencies) via the user interface of the server/central computer 30 without being in front of the physiological monitoring device 7.

Optionally or additionally, the server/central computer 30 may store the patient's physiological measurements and algorithms to provide recommended measurement configurations to users based on one or more of the patient's physiological parameters, medical history, and care area where the patient is currently located. For example, based on the patient's NIBP trends in a pre-determined time, the patient's medical history and/or the care area where the patient is located, the algorithms in the server/central computer 30 may provide recommended measurement configurations in adjusting NIBP measurement intervals and/or frequencies. Those in the art having the benefit of this disclosure will appreciate that the functionality of the server/central computer 30 may be distributed over a computing system such as a network or a cloud.

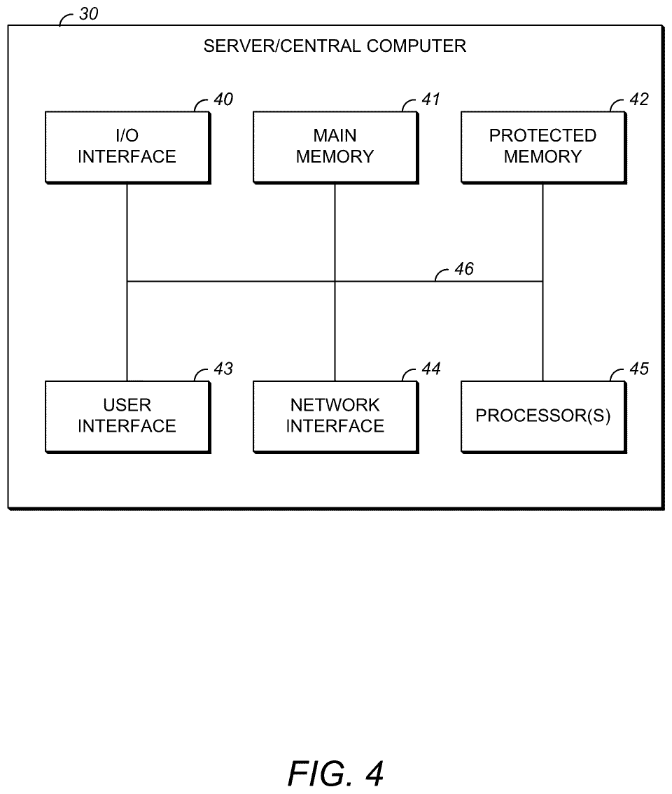

FIG. 4 is a schematic diagram of an example of a server/central computer according to an embodiment of the present disclosure. As shown in FIG. 4, the exemplary server/central computer 30 includes an I/O interface 40, a main memory 41, a protected memory 42, a user interface 43, a network interface 44, and one or more processors 45.

The I/O interface 40 can be implemented to accommodate various connections to the server/central computer 30 that include, but are not limited to, a universal serial bus (USB) connection, parallel connection, a serial connection, coaxial connection, a High-Definition Multimedia Interface (HDMI) connection, or other known connection in the art connecting to external devices. The I/O interface 40 can be an interface for enabling the transfer of information between server/central computer 30, one or more physiological monitoring devices 7, and external devices such as peripherals connected to the server/central computer 30 that need special communication links for interfacing with the one or more processors 45.

The main memory 41 can be used to store any type of instructions associated with algorithms, processes, or operations for controlling the general functions of the server/central computer 30 as well as any operating system such as Linux, UNIX, Windows Server, or other customized and proprietary operating systems.

The protected memory 42 is, for example, a processor reserved memory of dynamic random-access memory (DRAM) or other reserved memory module or secure memory location for storing more critical information such as confidential or proprietary patient information.

The user interface 43 is implemented for allowing communication between a user and the server/central computer 30. The user interface 43 includes, but is not limited to, a mouse, a keyboard, a liquid crystal display (LCD), thin film transistor (TFT), light-emitting diode (LED), high definition (HD) or other similar display device with touch screen capabilities. The network interface 44 is a software and/or hardware interface implemented to establish a connection between the server/central computer 30 and one or more physiological monitoring devices or other servers/central computer inside and outside the patient care or hospital environment.

It is contemplated by the present disclosure that network interface 44 includes software and/or hardware interface circuitry for establishing communication connections with the rest of the system 1a using both wired and wireless connections for establishing connections to, for example, a local area networks (LANs), wide area networks (WANs), metropolitan area networks (MANs) personal area networks (PANs), and wireless local area networks (WLANs), system area networks (SANs), and other similar networks.

The one or more processors 45 are used for controlling the general operations of the server/central computer 30. Communication between the components of the server/central computer 30 (e.g., 40-44) is established using an internal bus 46.

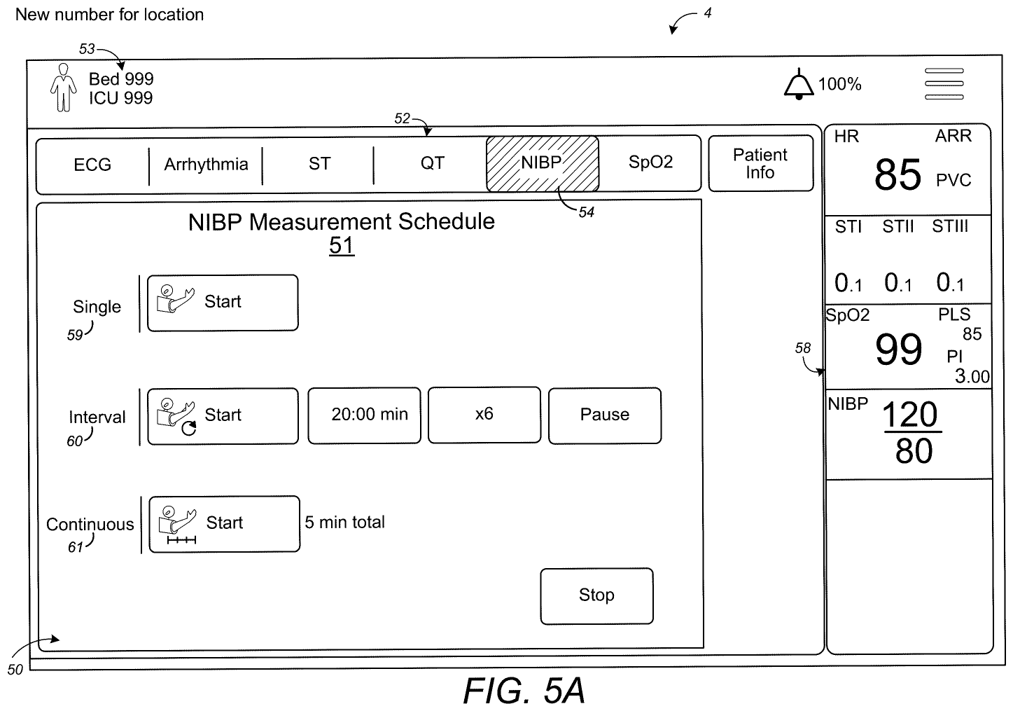

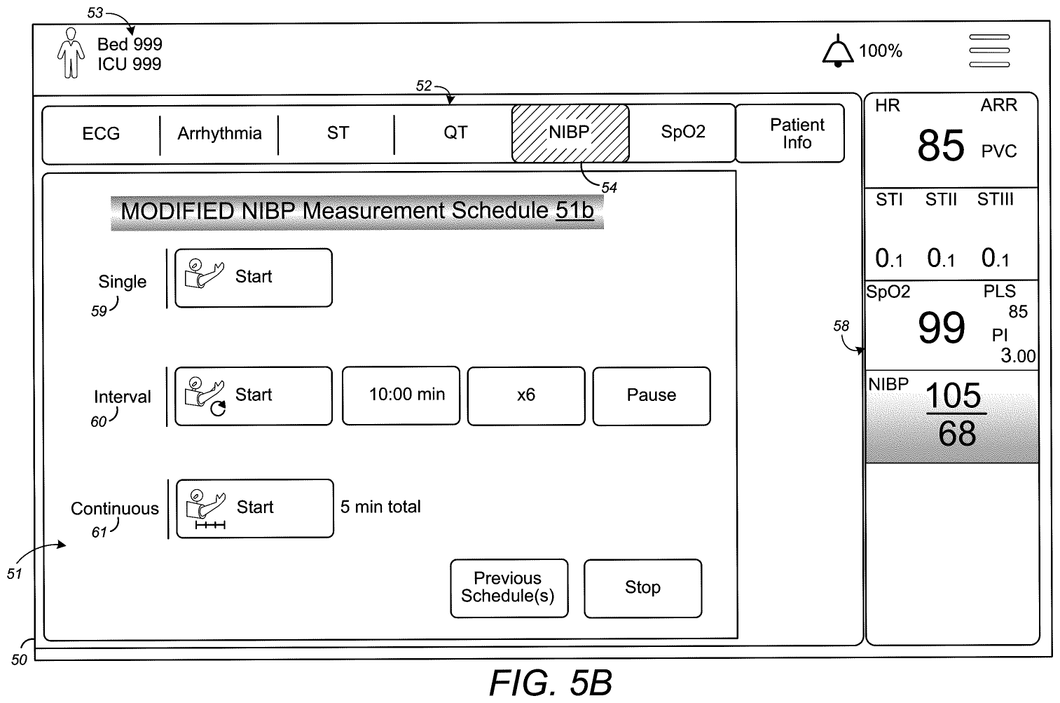

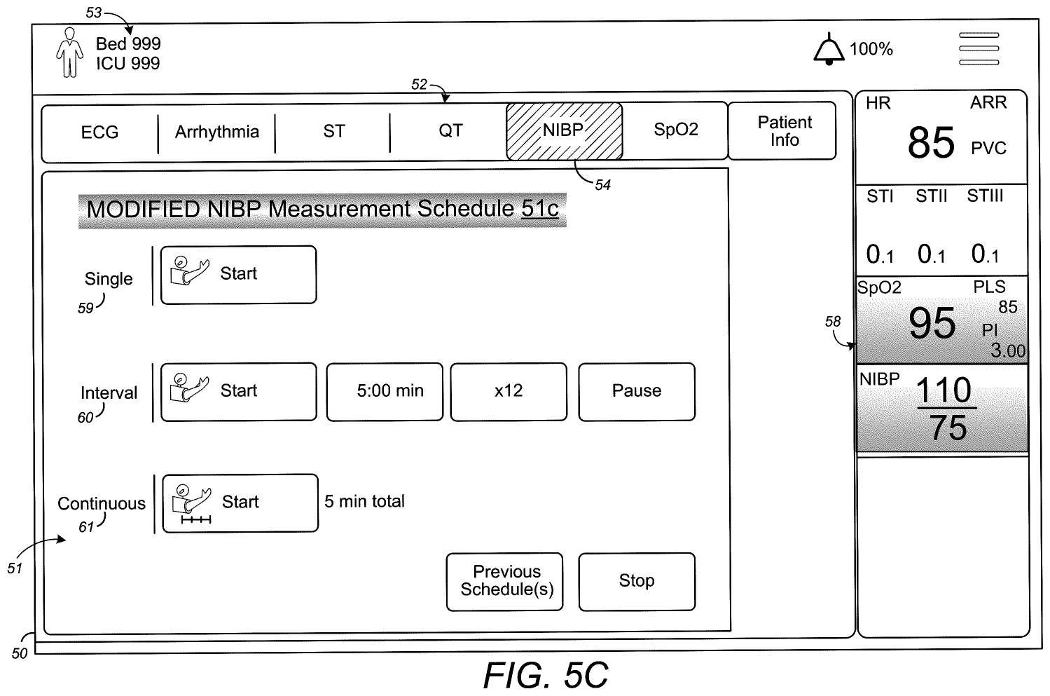

FIGS. 5-7 illustrate examples of graphical user interfaces (GUIs) displayed on the physiological monitoring device 7. The physiological monitoring device 7 is capable of executing both a predefined measurement schedule and a modified measurement schedule, which is automatically generated in response to changing patient parameters of the patient 1b. More specifically, the modified measurement schedule is automatically generated in response to a modification event, as detailed herein and according to the embodiments of the present disclosure.

It is contemplated by the present disclosure that the GUIs as shown in FIGS. 5-7 can be generated on the display 4 for allowing interaction with one or more users, by one or more processors 3 executing one or more programs stored in the memory 8 of an electronic device such as, but not limited to, a physiological monitoring device 7, as described with reference to FIGS. 1 and 2. Although the examples in FIGS. 5-7 refer to a physiological monitoring device 7, it is also contemplated by the present disclosure that the GUIs can be implemented on other electronic devices including, but not limited to, a hand-held computing device, a personal computer, an electronic tablet, a smartphone, or other similar hand-held electronic device capable of executing and displaying the GUI. For example, the GUIs as shown in FIGS. 5-7 can be implemented on the user interface 43 (e.g., display) of the server/central computer 30, such that users are allowed to control the functions of the physiological monitoring device 7 and the connected sensors 17.

While many parameters are often continuously monitored (e.g., ECG, SpO2), measurement schedules could be implemented in accordance with the systems and methods described herein. For instance, as shown in FIG. 5A, the GUI 50 provides several user-selectable inputs 52 for facilitating implementation of predefined measurement schedules for various physiological parameters such as ECG, Arrhythmia, ST segment, QT interval, NIBP, and SpO2, to list a few examples. Patient parameter information (e.g., HR, STI, STII, STII, SpO2, pulse, and NIBP) is displayed in parameter window 58. Additional parameters such as electroencephalogram (EEG), invasive blood pressures, blood glucose, temperature, blood pressure waveforms, pulse oximeter photoplethysmograph (Pleth or PPG) waveforms, and respiration parameters could also be displayed in the GUI 50 or parameter window 58.

In general, parameters that are measured in real-time are typically displayed in real-time, while parameters that are measured at periodic intervals may only display the most recently measured results. For example, cardiac information is typically measured continuously via one or more electrodes affixed to the patient 1b. Accordingly, this cardiac information is updated in real-time. Alternatively, NIBP is typically measured periodically at predefined intervals, which results in the displayed NIBP information being the results of the most recent measurement. If the NIBP is measured in real-time, then it would be displayed in real-time (see, for example, reference numeral 61 which permits continuous NIBP measurements).

The user-selectable inputs 52 for measuring various physiological parameters (e.g., ECG, Arrhythmia, ST segment, QT interval, NIBP, SpO2, and patient information) are provided as examples, and it is contemplated by the present disclosure that the user-selectable inputs 52 can include other parameters for scheduling additional and/or different physiological parameters, which are measured either discretely or continuously and displayed in parameter window 58. In FIG. 5A, the measured patient data is provided from, for example, the sensors 17 (e.g., monitoring various physiological parameters of the patent 1) to the physiological device 7 via the sensor interface 2.

As shown in the illustrated example, there are presently three measurement options: a single measurement 59 option, a predefined interval 60 option, and the continuous measurement 61 option. The NIPB input 54 has been selected in the illustrated embodiment. The single measurement 59 option performs a single NIBP measurement, and the results are displayed with the patient parameter window 58 until a more recent measurement is taken. The previous measurements are then stored in the memory 8 for later viewing and retrieval. In some embodiments, after a user-selectable and/or predetermined period of time, the measurement results are no longer displayed. This would help prevent medical personnel from relying on measurements that are outdated and possibly not an indication of the patient current status. Additionally, the parameter window 58 may be user-selectable to permit a user to see all the past measurements, which includes when the measurements were obtained. While the illustrated embodiment is directed toward NIBP, it is understood that similar scheduling features could be implemented for other parameters being measured by the physiological monitoring device 7.

The predefined interval 60 option will measure the NIBP at the predefined interval (e.g., every 20 minutes as illustrated) and for a predetermined number of times (e.g., 6 times as illustrated). These interval lengths can be default times or user-input parameters. Likewise, the schedule can be paused via the pause button or stopped altogether via a stop button. Lastly, a continuous measurement 61 option provides continuous measurement for a period of time (e.g., the patient's blood pressure is measured continuously for 5 minutes total). These continuous measurement lengths could be a default time, or user-entered lengths.

While the illustrated example shows intervals of 20 minutes, the intervals could be shorter or longer (e.g., as short as 1 minute, or as long as 8 hours or more between measurements). Likewise, the number of times the measurements are taken is also customizable. Similarly, the continuous measurement time could be shorter or longer than the illustrated 5 minutes.

Table 1 illustrates some examples of interval times and corresponding automatically generated interval times based on whether a current measured patient parameter is within, for example, a certain percentage of an alarm limit. Some non-limiting examples could include 5%, 10%, 12.5%, or 15% of the alarm limit. While, the table below is directed to NIBP measurement intervals, modified interval times could be generated for other patient parameters. Additionally, both the interval times and the modified interval times could be user-adjustable to permit users to adjust the intervals as needed. For example, different hospitals may have different standards and protocols for varying parameters. Likewise, as new medical information is learned within the healthcare industry, the times could be changed in order to update the measurement schedule and provide better health outcomes for patients.

TABLE 1

| Interval Time | | Modified Interval Time | |
|---|---|---|---|
| 1 | Minute | 1 | Minute |
| 2 | Minutes | 1 | Minute |
| 2.5 | Minutes | 1 | Minute |
| 3 | Minutes | 2 | Minutes |
| 5 | Minutes | 2.5 | Minutes |
| 10 | Minutes | 5 | Minutes |
| 15 | Minutes | 7 | Minutes |
| 20 | Minutes | 10 | Minutes |
| 25 | Minutes | 12 | Minutes |
| 30 | Minutes | 15 | Minutes |
| 45 | Minutes | 22 | Minutes |
| 60 | Minutes | 30 | Minutes |
| 120 | Minutes | 30 | Minutes |
| 240 | Minutes | 30 | Minutes |

FIG. 5B illustrates an example of a modification event, which caused a modified NIBP measure schedule 51b to be automatically generated and implemented. As illustrated, the patient parameter for NIBP has dropped (in this example: by approximately 12.5% from its original value). While the NIBP parameter has not yet exceeded any alarm thresholds (e.g., below 90 Systolic or 50 diastolic), the drop in NIBP could be an early indication of a potential medical issue. Even though the measured NIBP is still within a typical range (e.g., has not exceeded a threshold that would trigger an alarm), the schedule is modified such that NIBP measurements are now taken every 10 minutes. The illustrated example includes highlighted boxes to identify both that a modified schedule has been generated and implemented as well as which parameter caused the modified schedule change. To prevent alarm fatigue, the physiological monitoring device 7 may not highlight the information on the display. Rather, a message may be displayed on a central monitoring station or server/central computer 30 or a message may be sent to the medical personnel monitoring the patient. In alternative embodiments, the physiological monitoring device 7 may implement one or more automatic re-tests to determine if the measured change is simply the result of variance in testing or a potentially incorrect measurement.

Similarly, after implementation of the modified NIBP measurement schedule 51b, if the measured parameter returns to original and/or normal measurements (e.g., as defined by the medical personnel), the physiological monitoring device 7 could revert back to the original measurement schedule. Similarly, while the illustrated example shows an example of an NIBP parameter worsening and necessitating a shorter measurement interval, if the patient parameters are remaining in a normal range for a predefined period of time (or improving), then the physiological monitoring device 7 could implement a modified NIBP measurement schedule where the intervals become longer. See, for example, Table 2, hereinbelow.

TABLE 2

| Interval Time | | Modified Interval Time | |
|---|---|---|---|
| 30 | Minutes | 60 | Minutes |
| 45 | Minutes | 90 | Minutes |
| 60 | Minutes | 120 | Minutes |
| 2 | Hours | 4 | Hours |
| 4 | Hours | 6 | Hours |
| 6 | Hours | 8 | Hours |
| 8 | Hours | 12 | Hours |

FIG. 5C provides an example of how the physiological monitoring device 7 can automatically generate more frequent intervals in response to multiple parameters dropping slightly. In this example, both the SpO2 and NIBP values are within typically normal ranges. However, two parameters have dropped slightly (as compared to FIGS. 5A and 5B). In this scenario, the physiological monitoring device 7 automatically increases the frequency of the measurements (e.g., from twenty-minute intervals to five-minute intervals) and also increases the length of time for the measurements to be taken (e.g., two hours instead of one hour).

As detailed previously, if patient parameters are remaining in a normal or healthy range for a predefined period of time, then the physiological monitoring device 7 could return to the original schedule and/or return to a modified NIBP measurement schedule where the intervals are greater.

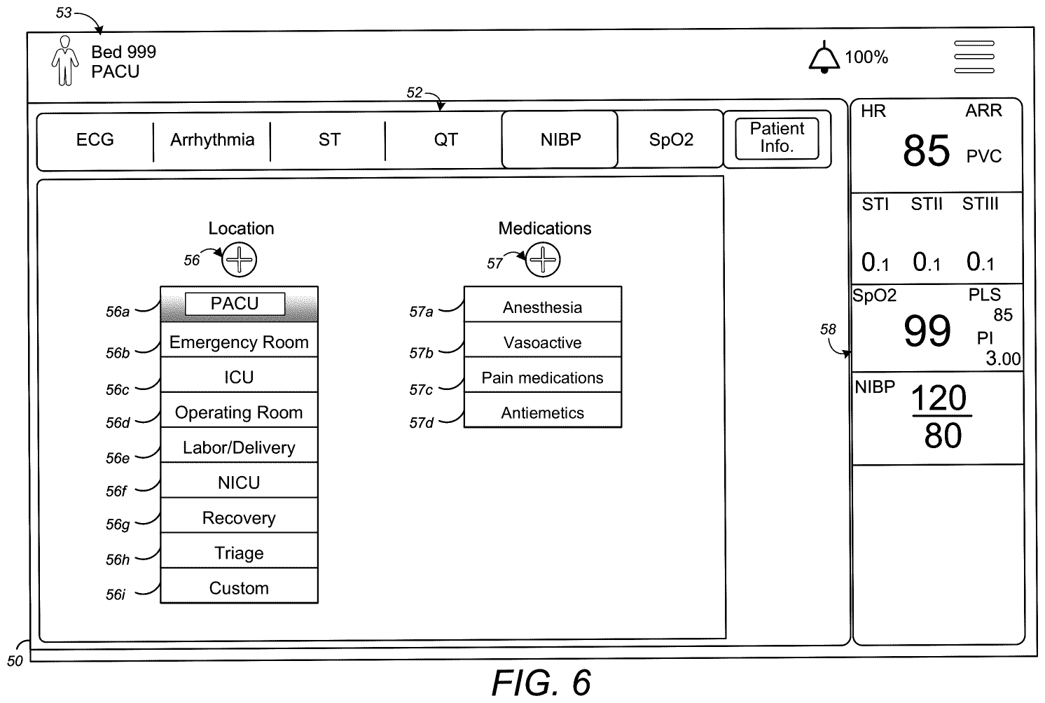

FIG. 6 illustrates a user interface for entering additional information about patient medication and patient location, which helps reduce the time that clinical providers spend configuring various settings. Likewise, the patient and location information can then be used during the automatic generation of the modified measurement schedules. While the illustrated embodiment shows information being manually entered, this information could also be obtained from an electronic medical record (EMR) database that is communicatively coupled to the patient monitor.

In the illustrated embodiment, a user selects the location selection button 56 and the GUI 50 provides various user-selectable location options. The additional options 56a-56i can be provided as, for example, a drop-down menu or a similar list of selectable location options. As illustrated, some exemplary locations include Post Anesthesia Care Unit (PACU) 56a, Emergency Room 56b, Intensive Care Unit (ICU) 56c, Operating Room 56d, labor and delivery rooms 56e, Natal Intensive Care Unit (NICU) 56f, Recovery Room 56g, Triage Room 56h, and/or a custom location 56i, to list a few examples. These pre-defined locations (e.g., 56a-56i) shown in FIG. 6 are merely examples and it is contemplated by the present disclosure that other additional pre-defined locations could be included. As illustrated, PACU 56a, has been selected and location information 53 is updated to reflect the user-entered location change.

In some embodiments not shown, location information may instead be pushed to or pulled from an electronic medical record (EMR) using one or more of the communications capabilities discussed above. The EMR may be stored, for example, on the server/central computer 30. If the information is pushed to the EMR, the physiological monitoring device 7 can use one or more location capabilities such as location data system 26, shown in FIG. 2, to acquire an absolute position. The absolute position can then be mapped onto a representation of the facility to determine where the physiological monitoring device is located within the facility.

Based on the selected location, the device location information 53 will be updated to reflect the selected location. Additionally, or alternatively, this location information may be obtained via information that is received by the physiological monitoring device 7 from the monitor mount 10, which is programmed with location information. In another embodiment, the location information could be received wirelessly from transmitters located throughout the medical facility. The transmitters could be, for example, wireless access points, or RFID transmitters (radio frequency identification), which communicate with RFID tags installed within the physiological monitoring device 7.

One reason for adjustable location information is because patients that are in different stages of their care and recovery likely need different levels of attention and care. For example, a patient in an ICU room may need more frequent NIBP measurements, whereas a patient in a recovery room may need fewer NIBP measurements. Additionally, a patient that has been improving health-wise, may need even fewer NIBP measurements as their health improved over time. Likewise, when the patient 1b is moved from a first location to a second location different from the first location, the measurement schedule may need to be further adjusted automatically to account for this movement (e.g., as a patient is moved from an ER room to a recovery room).

Similarly, a user may enter medications 57a-57d being administered to the patient. Based on the medications the patient is taking, the physiological monitoring device 7 may modify the measurement schedule (e.g., a patient waking up from anesthesia, a patient on blood thinners, and/or a patient on vasodilators may need additional monitoring. Some examples of vasodilators, which may decrease blood pressure, include Nitroprusside, Nitroglycerin, Hydralazine, and Nicardipine. Some examples of vasopressor, which may increase blood pressure, include Dopamine, Epinephrine, Norepinephrine, Vasopressin, and Neosynephrine. Some examples of pain control medications may include Morphine, hydromorphone, hydrocodone and meperidine for pain control. Likewise, antiemetics like dexamethasone cause also changes in blood pressure, which needed to be accounted for. Lastly, some combinations of narcotics and anxiolytics can cause a drop in blood pressure. Thus, it is important to be aware of these potentials for drops in blood pressure. Both from a clinical standpoint to ensure a patient is healthy, but also when measuring patient parameters to account for factors that might cause a change in blood pressure (or other patient parameter.

In some embodiments not shown, medication information may instead be pulled from an electronic medical record (EMR) using one or more of the communications capabilities discussed above. The EMR may be stored, for example, on the server/central computer 30. The physiological monitoring device 7 in these embodiments may also pull patient care information associated with the medications in the medication information from, for example, one or more databases or other data structures. The physiological monitoring device 7 may also be associated with various electronic inventories stored in one or more data structures and used to track certain medications as described above. The data structures may also be stored, for example, on the sever/central computer 30.

Additional patient information such as height, weight, sex, and age may also be entered here to provide additional relevant information that can be used to automatically generate a modified measurement schedule 51d. For example, the modified measurement schedule could be further adjusted based on whether a patient is an infant in the NICU, a teenager, an adult, or is elderly.

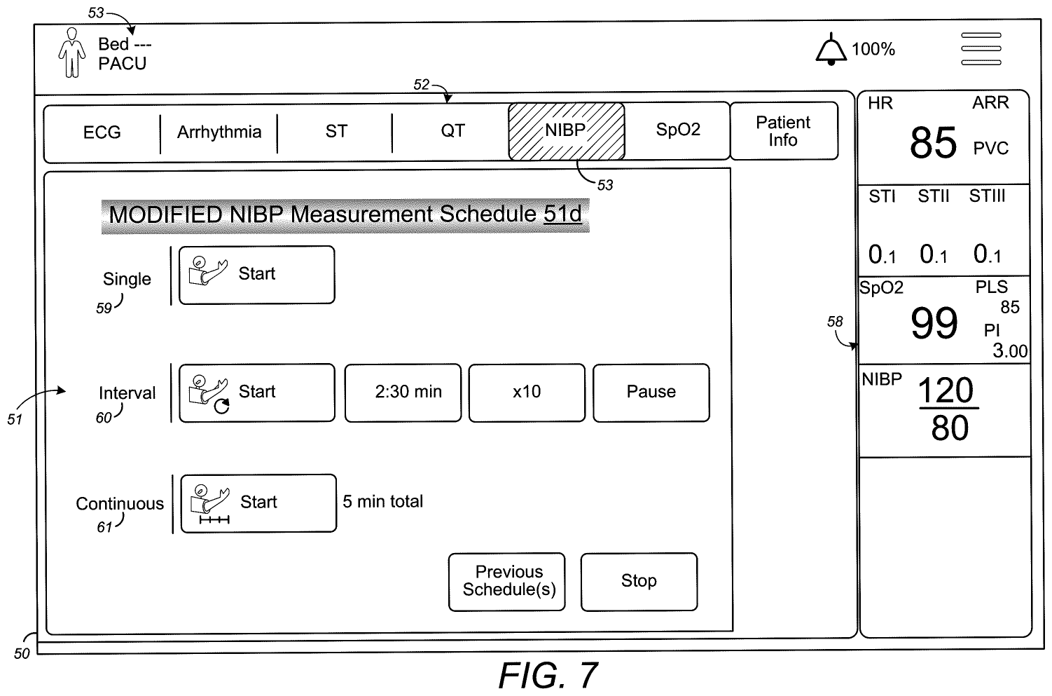

FIG. 7 illustrates how the physiological monitoring device 7 automatically updates the measurement schedule and location information 53 based on the updated location and medication information.

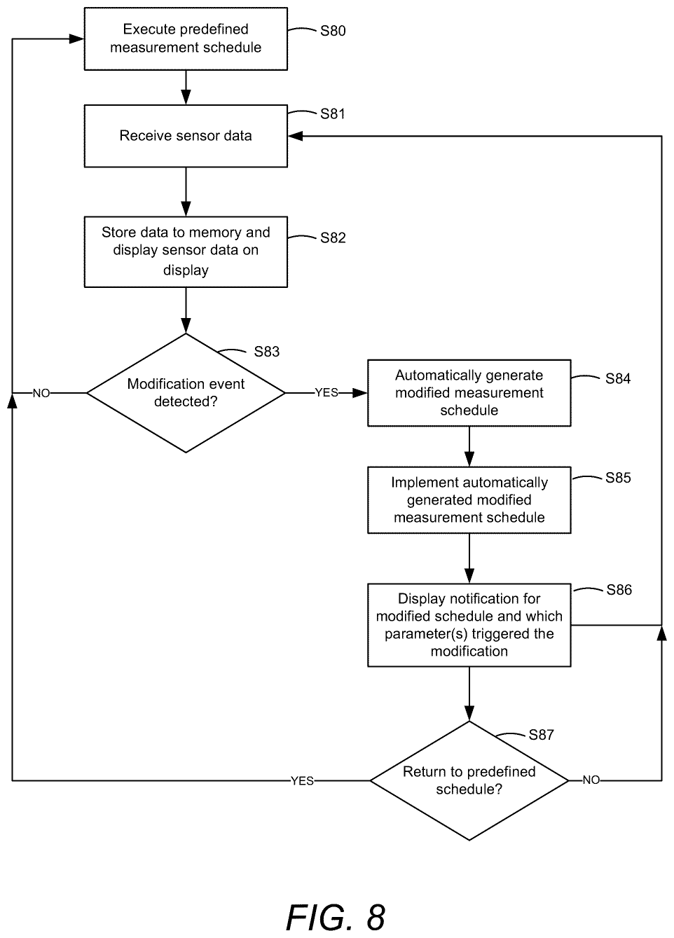

The method and algorithm described in FIG. 8 describe steps performed by the physiological monitoring device 7 when implementing a modified measurement schedule 51b-d.

In a first step S80, the physiological monitoring device 7 executes a predefined measurement schedule based on the patient current health. The execution of the predefined measurement schedule causes the physiological monitoring device 7 to receive signals and/or sensor data from one or more sensors affixed to the patient 1b in step S81. These received signals and/or sensor data are then stored in the memory 8 of the physiological monitoring device 7 and displayed on a display 4 in step S82.

In the next step S83, the physiological monitoring device 7 determines a modification event has occurred based on the received signals and/or sensor data being. For example, a modification event could be triggered in response to one or more of a change in the medical condition of the patient, a change in the location of the patient, and a change in the personal and/or physiological information of the patient. Some examples of changes in the medical condition of the patient include hypovolemia, sepsis, cardiac events, and shock. Similarly, a change in the location of the patient may include the patient moving to an emergency room, an operating room, an intensive care unit, a natal intensive care unit, post-anesthesia care unit (PACU), a recovery room, a labor and delivery room, Triage, or some other custom location entered by the user. Additionally, a change in personal and/or physiological information of the patient may include a user obtaining information about at least one of the current medications taken or administered to the patient, information about the patient's medical history, and personal information including details about the patient's age, height, weight, and/or gender. Some examples of relevant medications may include anesthesia, vasoactive medications, and pain medications. Lastly, in some embodiments, the modification event is based on a percentage change in the one or more of the monitored patient parameters and/or one or more of the monitored patient parameters being within a certain percentage of an alarm limit.

If a modification event is not detected, then the physiological monitoring device 7 returns to step S80 to continue to execute the predefined measurement schedule. If, however, the physiological monitoring device 7 does detect a modification event, then the physiological monitoring device 7 will automatically generate a modified measurement schedule (as detailed, for example, in FIGS. 5B, 5C, and FIG. 7) in S84. Likewise, in S85 and S86, the physiological monitoring device 7 also automatically implements the modified measurement schedule, and displays a notification that a modified schedule is implemented, and indicates which parameter(s) triggered the modification, respectively.

Lastly, in S87, the physiological monitoring device 7 determines whether to return to execute the predefined measurement schedule. The physiological monitoring device 7 may return to the predefined measurement schedule in response to a user canceling or overriding the modified schedule or due to a patient's health improving such that the modified schedule is no longer needed, list a couple of examples.

The present disclosure may be implemented as any combination of an apparatus, a system, an integrated circuit, and a computer program on a non-transitory computer-readable recording medium. The one or more processors may be implemented as an integrated circuit (IC), an application-specific integrated circuit (ASIC), or large-scale integrated circuit (LSI), system LSI, super LSI, or ultra LSI components which perform a part, or all of the functions described in the present disclosure. The one or more processors, for example, processor(s) 3 and processor(s) 12 in FIG. 1, microcontrollers 3a and 3b in FIG. 2, and processor(s) in FIG. 4 can be, but are not limited to, a central processing unit (CPU), a hardware microprocessor, a multi-core processor, a single core processor, a field programmable gate array (FPGA), a microcontroller, an application specific integrated circuit (ASIC), a digital signal processor (DSP), or other similar processing device capable of executing any type of instructions, algorithms, or software for controlling the operation, and performing the functions of e.g., the physiological monitoring device 7 (as illustrated in FIGS. 1 and 2) and the monitor mount 10 (as illustrated in FIG. 1) and the server/central computer 30 (as illustrated in FIG. 4).

The present disclosure includes the use of computer programs or algorithms. The programs or algorithms can be stored on a non-transitory computer-readable medium for causing a computer, such as the one or more processors, to execute the functions and steps as described with reference to FIGS. 5-8. For example, the memories 8 and 13 in FIG. 1, the memory 8 in FIG. 2, and the main memory 41 in FIG. 4 can be a single memory or one or more memories or memory locations that include, but are not limited to, a random access memory (RAM), a memory buffer, a hard drive, a database, an erasable programmable read only memory (EPROM), an electrically erasable programmable read only memory (EEPROM), a read only memory (ROM), a flash memory, hard disk or any other various layers of memory hierarchy. For example, the one or more memories stores software or algorithms with executable instructions and the one or more processors can execute a set of instructions of the software or algorithms in association with generating, displaying, customizing, and executing measurement schedules on a GUI for measuring physiological parameters of patients, as described with reference to FIGS. 5-8.

The computer programs, which can also be referred to as programs, software, software applications, applications, components, or code, include machine instructions for a programmable processor, and can be implemented in a high-level procedural language, an object-oriented programming language, a functional programming language, a logical programming language, or an assembly language or machine language. The term computer-readable recording medium refers to any computer program product, apparatus, or device, such as a magnetic disk, optical disk, solid-state storage device, memory, and programmable logic devices (PLDs), used to provide machine instructions or data to a programmable data processor, including a computer-readable recording medium that receives machine instructions as a computer-readable signal.

By way of example, a computer-readable medium can comprise DRAM, RAM, ROM, EEPROM, CD-ROM or other optical disk storage, magnetic disk storage or other magnetic storage devices, or any other medium that can be used to carry or store desired computer-readable program code in the form of instructions or data structures and that can be accessed by a general-purpose or special-purpose computer, or a general-purpose or special-purpose processor. Disk or disc, as used herein, include compact disc (CD), laser disc, optical disc, digital versatile disc (DVD), floppy disk, and Blu-ray disc where disks usually reproduce data magnetically, while discs reproduce data optically with lasers. Combinations of the above are also included within the scope of computer-readable media.

Use of the phrases “capable of,” “capable to,” “operable to,” or “configured to” in one or more embodiments, refers to some apparatus, logic, hardware, and/or element designed in such a way to permit use of the apparatus, logic, hardware, and/or element in a specified manner. The subject matter of the present disclosure is provided as examples of apparatus, systems, methods, and programs for performing the features described in the present disclosure. However, further features or variations are contemplated in addition to the features described above. It is contemplated that the implementation of the components and functions of the present disclosure can be done with any newly arising technology that may replace any of the above implemented technologies.

Although specific visual indications are described with reference to FIGS. 5-10 (e.g., check mark, etc.), it is contemplated by the present disclosure that almost any visual indication can be implemented that effectively conveys the status of any measurement schedule and other aspects of the GUI 50 to the user. Additionally, the above description of “selection” or “selections” as described with reference to FIGS. 5-7 (e.g., “start”, “stop”, etc.) are examples of virtual tab, buttons, icons, labels, or other selectable symbols within the GUI 50 that allow interaction between the user and the GUI 50.

Additionally, the above description provides examples, and is not limiting of the scope, applicability, or configuration set forth in the claims. Changes may be made in the function and arrangement of elements discussed without departing from the spirit and scope of the disclosure. Various embodiments may omit, substitute, or add various procedures or components as appropriate. For instance, features described with respect to certain embodiments may be combined in other embodiments.

Various modifications to the disclosure will be readily apparent to those skilled in the art, and the generic principles defined herein may be applied to other variations without departing from the spirit or scope of the present disclosure. Throughout the present disclosure the terms “example,” “examples,” or “exemplary” indicate examples or instances and do not imply or require any preference for the noted examples. Thus, the present disclosure is not to be limited to the examples and designs described herein but is to be accorded the widest scope consistent with the principles and novel features disclosed.

1. A medical device configured to automatically generate customized measurement schedules, the medical device comprising:
one or more sensors attached to a patient configured to measure patient parameters of the patient according to a predefined measurement schedule;
one or more processors;
a memory configured to store one or more programs that are executed by the one or more processors; and
a display configured to display the measured patient parameters;
wherein the one or more processors are communicatively connected to the memory and configured to execute the one or more programs to:
execute the predefined measurement schedule configured to activate the one or more sensors in order to measure the patient parameters of the patient;
receive the measured patient parameters from the one or more sensors;
transmit the received measured patient parameters to memory and to the display;
generate a modified measurement schedule automatically in response to a modification event detected from the received measured patient parameters;
implement the modified measurement schedule; and
provide an indication that modified measurement schedule has been generated and implemented.

2-6. (canceled)

7. The medical device of claim 1, wherein the modification event is based on a percentage change in one or more of the measured patient parameters.

8. The medical device of claim 1, wherein the modification event is based on one or more of the measured patient parameters being within a predefined percentage of an alarm limit.

9. The medical device of claim 1, wherein patient parameters of the patient include at least one of non-invasive blood pressure (NIBP), temperature, heart rate, an electrocardiogram (ECG), non-invasive peripheral oxygen saturation (SpO2), end tidal carbon dioxide (etCO2), apnea of the patient, neuromuscular transmission (NMT), and cardiac output (CO).

10. The medical device of claim 1, wherein providing a notification that a modified measurement schedule has been automatically generated and implemented includes providing a notification by at least one of an electronic mail, a text message, a page message sent to a pager, a notification being transmitted to a central monitoring station, and an alert displayed on the medical device.

11. The medical device of claim 10, wherein the notification includes at least one of a visual and an audible notification indicating generation and implementation of the modified measurement schedule.

12. The medical device of claim 1, wherein the one or more processors stores in the memory a time, date, and the modification event that caused the generation and implementation of the automatically generated modified measurement schedule.

13-16. (canceled)

17. A method for automatically generating customized measurement schedules by a medical device, the method comprising:
executing a predefined measurement schedule configured to activate the one or more sensors in order to measure the patient parameters of the patient;
receiving the measured patient parameters from the one or more sensors;
transmitting the received measured patient parameters to memory and to the display;
generating a modified measurement schedule automatically in response to a modification event detected from the received measured patient parameters;
implementing the modified measurement schedule; and
providing an indication that modified measurement schedule has been generated and implemented.

18. The method of claim 17, wherein the modification event includes at least one of a change in a medical condition of the patient, a change in a location of the patient, a change in personal information of the patient, and a change in physiological information of the patient.

19. The method of claim 18, wherein the change in a location of the patient includes at least one of the patient moving to an emergency room, an operating room, an intensive care unit, a neonatal intensive care unit, a post anesthesia care unit, a recovery room, and a labor and delivery room.

20. The method of claim 17, wherein the modification event is based on a percentage change in one or more of the measured patient parameters.

21. The method of claim 17, wherein the modification event is based on one or more of the measured patient parameters being within a predefined percentage of an alarm limit.

22. The method of claim 17, wherein patient parameters of the patient include at least one of non-invasive blood pressure (NIBP), temperature, heart rate, an electrocardiogram (ECG), non-invasive peripheral oxygen saturation (SpO2), end tidal carbon dioxide (etCO2), apnea of the patient, neuromuscular transmission (NMT), and cardiac output (CO).

23. (canceled)

24. A medical system, comprising:
a monitor mount; and
a medical device mounted to the monitor mount and configured to automatically generate customized measurement schedules, the medical device comprising:
one or more sensors attached to a patient configured to measure patient parameters of the patient according to a predefined measurement schedule;
one or more processors;
a memory configured to store one or more programs that are executed by the one or more processors; and
a display configured to display the measured patient parameters;
wherein the one or more processors are communicatively connected to the memory and configured to execute the one or more programs to:
execute the predefined measurement schedule configured to activate the one or more sensors in order to measure the patient parameters of the patient;
receive the measured patient parameters from the one or more sensors;
transmit the received measured patient parameters to memory and to the display;
generate a modified measurement schedule automatically in response to a modification event detected from the received measured patient parameters;
implement the modified measurement schedule; and
provide an indication that modified measurement schedule has been generated and implemented.

25. The medical system of claim 24, wherein the modification event includes at least one of a change in a medical condition of the patient, a change in a location of the patient, a change in personal information of the patient, and a change in physiological information of the patient.

26. The medical system of claim 25, wherein the change in the medical condition of the patient includes at least one of detection hypovolemia, sepsis, cardiac events, and shock.

27. The medical system of claim 25, wherein the change in a location of the patient includes at least one of the patient moving to an emergency room, an operating room, an intensive care unit, a neonatal intensive care unit, a post anesthesia care unit, a recovery room, and a labor and delivery room.

28. The medical system of claim 25, wherein the change in personal information of the patient includes obtaining information about at least one of current medications taken by or administered to the patient, information about the patient's medical history, and personal information including information about the patient's age, height, weight, and/or gender.

29. The medical system of claim 24, wherein the modification event is based on:
a percentage change in one or more of the measured patient parameters; or
one or more of the measured patient parameters being within a predefined percentage of an alarm limit; or
a combination thereof.

30. (canceled)

31. The medical system of claim 24, wherein patient parameters of the patient include at least one of non-invasive blood pressure (NIBP), temperature, heart rate, an electrocardiogram (ECG), non-invasive peripheral oxygen saturation (SpO2), end tidal carbon dioxide (etCO2), apnea of the patient, neuromuscular transmission (NMT), and cardiac output (CO).
